(19) United States
(12) Patent Application Publication
(10) Pub. No.: US 2025/0213307 A1
(43) Pub. Date: Jul. 3, 2025

(54) IMPLANT IDENTIFICATION

(71) Applicant: Paragon 28, Inc., Englewood, CO (US)

(72) Inventor: Luciano Bernardino BERTOLOTTI, Denver, CO (US)

(73) Assignee: Paragon 28, Inc., Englewood, CO (US)

(21) Appl. No.: 18/985,743

(22) Filed: Dec. 18, 2024

Related U.S. Application Data
(63) Continuation of application No. PCT/US2022/073135, filed on Jun. 24, 2022.

(51) Int. Cl. A61B 34/10; G06T 7/00; G16H 30/40

(52) U.S. Cl. CPC A61B 34/10; G06T 7/0012; G16H 30/40; A61B 2034/105; A61B 2034/108; G06T 2207/20081

(57) ABSTRACT

Implant identification includes obtaining a machine learning (ML) model trained to select, based on properties of anatomical surfaces of anatomy adjacent to subject anatomy to be replaced, anatomy implant models of physical implants for replacement of the subject anatomy, obtaining imaging data of an anatomical region of a patient, the anatomical region comprising a subject anatomy and other anatomy adjacent to the subject anatomy, determining properties of anatomical surface(s) of the other anatomy, the anatomical surface(s) being at a respective interface(s) between the other anatomy and the subject anatomy, and applying the ML model, using the determined properties of the anatomical surface(s), and obtaining a selected implant model selected by the ML model as a specification of a physical implant for potential surgical implantation at least partially within the patient as a replacement of the subject anatomy.

### BACKGROUND

Some anatomical injuries can be more difficult to heal than others. For example, various bones lack a blood supply ample enough to facilitate rapid healing, thereby rendering a patient potentially unable to perform certain physical functions for a prolonged healing period. In some cases, particularly with certain bones of the human body, injuries can be addressed by an implant device to replace the bone or portion thereof. Due to interactions between such a bone and other anatomy, for example other bones in the case of joints, accurate identification, selection, and creation of the appropriate implant to use for a given patient is important.

### SUMMARY

Shortcomings of the prior art are overcome and additional advantages are provided through the provision of a computer-implemented method. The method obtains a machine learning model trained to select, based on properties of anatomical surfaces of anatomy that is adjacent to subject anatomy to be partially or totally replaced, anatomy implant models of physical implants for partial or total replacement of the subject anatomy; obtains imaging data of an anatomical region of a patient, the anatomical region comprising a subject anatomy of the patient and other anatomy of the patient, the other anatomy being adjacent to the subject anatomy of the patient; determines, from the imaging data, properties of at least one anatomical surface of the other anatomy, the at least one anatomical surface being at a respective at least one interface between the other anatomy and the subject anatomy of the patient; and applies the machine learning model, using the determined properties of the at least one anatomical surface, and obtaining, based on the applying, a selected implant model, the selected implant model being selected by the machine learning model as a specification of a physical implant for potential surgical implantation at least partially within the patient as a partial or total replacement of the subject anatomy of the patient.

Further, a computer system is provided that includes a memory and a processor in communication with the memory, wherein the computer system is configured to perform a method. The method obtains a machine learning model trained to select, based on properties of anatomical surfaces of anatomy that is adjacent to subject anatomy to be partially or totally replaced, anatomy implant models of physical implants for partial or total replacement of the subject anatomy; obtains imaging data of an anatomical region of a patient, the anatomical region comprising a subject anatomy of the patient and other anatomy of the patient, the other anatomy being adjacent to the subject anatomy of the patient; determines, from the imaging data, properties of at least one anatomical surface of the other anatomy, the at least one anatomical surface being at a respective at least one interface between the other anatomy and the subject anatomy of the patient; and applies the machine learning model, using the determined properties of the at least one anatomical surface, and obtaining, based on the applying, a selected implant model, the selected implant model being selected by the machine learning model as a specification of a physical implant for potential surgical implantation at least partially within the patient as a partial or total replacement of the subject anatomy of the patient.

Yet further, a computer program product including a computer readable storage medium readable by a processing circuit and storing instructions for execution by the processing circuit is provided for performing a method. The method obtains a machine learning model trained to select, based on properties of anatomical surfaces of anatomy that is adjacent to subject anatomy to be partially or totally replaced, anatomy implant models of physical implants for partial or total replacement of the subject anatomy; obtains imaging data of an anatomical region of a patient, the anatomical region comprising a subject anatomy of the patient and other anatomy of the patient, the other anatomy being adjacent to the subject anatomy of the patient; determines, from the imaging data, properties of at least one anatomical surface of the other anatomy, the at least one anatomical surface being at a respective at least one interface between the other anatomy and the subject anatomy of the patient; and applies the machine learning model, using the determined properties of the at least one anatomical surface, and obtaining, based on the applying, a selected implant model, the selected implant model being selected by the machine learning model as a specification of a physical implant for potential surgical implantation at least partially within the patient as a partial or total replacement of the subject anatomy of the patient.

Additionally or alternatively, the method can include providing the selected implant model to a candidate model specification module for specification of a candidate model for validation.

Additionally alternatively, the method can include presenting the selected implant model to a user on a graphical user interface; receiving manipulations to the selected implant model, the manipulations changing the selected implant model and producing the candidate implant model for validation, wherein the candidate implant model specifies a physical implant with a different one or more physical properties in comparison to the physical implant specified by the selected implant model; and providing the candidate implant model to a validation module for validation, the validation determining whether the physical implant specified by the candidate implant model passes for surgical implantation within the patient.

Additionally or alternatively, the manipulations to the selected implant model can include: at least one manipulation specified by the user and/or at least one manipulation determined automatically by artificial intelligence.

Additionally or alternatively, based on the validation determining that the physical implant specified by the candidate implant model does not pass, the method can include iterating, one or more times, (i) the receiving manipulations and (ii) the providing the candidate implant model to the validation module for validation, wherein at each iteration of the iterating, the candidate implant model that did not pass is provided as the selected implant model for a next iteration of the iterating.

Additionally or alternatively, the method can include receiving manipulations to the properties of the at least one anatomical surface of the other anatomy, wherein the validation to determine whether the physical implant specified by the candidate implant model passes for surgical implantation within the patient is based at least in part on the manipulated properties of the at least one anatomical surface.

Additionally or alternatively, the manipulations to the properties of the at least one anatomical surface can include at least one manipulation specified by the user and/or at least one manipulation determined automatically by artificial intelligence.

Additionally or alternatively, the method can include receiving manipulations to the properties of the at least one anatomical surface of the other anatomy, and providing the selected implant model, as the candidate implant model for validation, to a validation module for validation, the validation determining whether the physical implant specified by the candidate implant model passes for surgical implantation within the patient, wherein the validation is based at least in part on the manipulated properties of the at least one anatomical surface.

Additionally or alternatively, based on determining the candidate implant model after receiving (i) manipulations to the selected implant model, the manipulations changing the selected implant model and producing the candidate implant model for validation, wherein the different candidate implant model specifies a physical implant with a different one or more physical properties in comparison to the physical implant specified by the selected implant model and/or (ii) manipulations to the properties of the at least one anatomical surface of the other anatomy, the method can include providing the candidate implant model to a validation module for validation, the validation determining whether the physical implant specified by the candidate implant model passes for surgical implantation within the patient, and based on the validation determining that the physical implant specified by the candidate implant model passes for surgical implantation within the patient, indicating the candidate implant model in a training dataset as part of a training example that correlates the candidate implant model to the at least one anatomical surface of the other anatomy.

Additionally or alternatively, the method can include providing the selected implant model, as a candidate implant model, to a validation module for validation, the validation determining whether the physical implant specified by the candidate implant model passes for surgical implantation within the patient.

Additionally or alternatively, the candidate implant model can be an initial candidate implant model. Based on the validation determining that the physical implant specified by the initial candidate implant model does not pass, the method can include (a) receiving (i) manipulations to the initial candidate implant model, the manipulations changing the initial candidate implant model and producing a different candidate implant model for validation, wherein the different candidate implant model specifies a physical implant with a different one or more physical properties in comparison to the physical implant specified by the initial candidate implant model and/or (ii) manipulations to the properties of the at least one anatomical surface of the other anatomy, (b) determining a next candidate implant model to provide to the validation module for validation, wherein based on receiving manipulations to the initial candidate implant model, the next candidate implant model is determined to be the different implant model produced from the manipulations to the initial candidate implant model, or based on receiving manipulations to the properties of the at least one anatomical surface of the other anatomy and not receiving manipulations to the initial candidate implant model, the next candidate implant model is determined to be the initial candidate implant model, and (c) providing the next candidate implant model to the validation module for validation to determine whether the physical implant specified by the next candidate implant model passes for surgical implantation within the patient, wherein the validation to determine whether the physical implant specified by the next candidate implant model passes for surgical implantation within the patient is based at least in part on the received at least one selected from the group consisting of the manipulations to the initial candidate implant model and the manipulations to the properties of the at least one anatomical surface.

Additionally or alternatively, the method can include training the machine learning model to select the anatomy implant models, wherein the training uses samples from a library of implant models and trains the machine learning model to select the anatomy implant models from the library of implant models, and wherein the selected implant model is selected by the machine learning model from the library of implant models.

Additionally or alternatively, the machine learning model can include a trained generator of a generative adversarial network (GAN), wherein the generator is trained using samples from a library of implant models, and is trained to generate implant models, wherein the selected implant model comprises an implant model generated by the generator and selected by the generator for output as the selected implant model.

Additionally or alternatively, the obtained imaging data can include three-dimensional digital model data representing the anatomical region of the patient, and the determining the properties of at least one anatomical surface of the other anatomy can include: processing the imaging data to present the at least one anatomical surface as at least one digital three-dimensional surface; and converting the at least one digital three-dimensional surface to at least one two-dimensional projection, wherein the determined properties of the at least one anatomical surface are determined from the at least one two-dimensional projection.

Additionally or alternatively, the method can include preprocessing the imaging data to produce a three-dimensional digital model of the anatomical region of the patient with the subject anatomy omitted therefrom.

Additionally or alternatively, the one or more anatomical surfaces can include one or more articular surfaces with which the physical implant is to engage based on being surgically implanted at least partially within the patient.

Additionally or alternatively, the anatomical region can include a patient ankle, wherein the subject anatomy comprises a talus, and wherein the at least one anatomical surface comprises at least one articular surface of at least one bone adjacent to the talus.

Additionally or alternatively, the determining the properties of the at least one anatomical surface can be based on (i) manual indication, by a user, of the at least one anatomical surface provided based on user input to a graphical user interface displaying a model comprising at least the other anatomy of the patient and/or (ii) automated analysis of the imaging data to ascertain the at least one anatomical surface.

Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 illustrates an example environment to incorporate and use aspects described herein;

FIGS. 2A-2C depict example segmented computed tomography (CT) images in accordance with aspects described herein;

FIGS. 3A-3B present an example ankle model in which a talus bone is omitted;

FIGS. 4A-4B depict an example bone model showing identified articular surfaces in accordance with aspects described herein;

FIGS. 5A-5B depict properties of articular surfaces isolated from patient anatomy, in accordance with aspects described herein;

FIG. 6 depicts an example interface for view and selection of a best-fit implant model in accordance with aspects described herein;

FIG. 7 depicts a conceptual illustration of machine learning model training and use in accordance with aspects described herein;

FIG. 8 depicts an example process flow for artificial intelligence-based implant selection, in accordance with aspects described herein;

FIG. 9 depicts an example graphical user interface of software for performing aspects described herein;

FIG. 10 depicts an example command line interface identifying model results;

FIG. 11 depicts an example interface for view of candidate best-fit implant models in accordance with aspects described herein;

FIG. 12 depicts an example process for implant selection, in accordance with aspects described herein; and

FIG. 13 depicts one example of a computer system and associated devices to incorporate and/or use aspects described herein.

### DETAILED DESCRIPTION

Described herein are approaches for anatomical analysis and optimal anatomy implant hardware selection. Aspects can identify optimal anatomy implant models based on existing patient anatomy. In particular embodiments, a machine learning (ML) model, such as a deep neural network, is trained to select at least one optimal or ‘best-fit’ anatomy implant model and/or characteristics thereof based on one or more surface(s) of surrounding patient anatomy with which the corresponding physical implant is expected to engage or interact once provided within the patient. By way of specific example, artificial intelligence (AI)-powered optimal talus implant model selection is provided, in which software identifies from a database of talus models one or more best-fit such models for a talus implant. An identified implant model can inform the selection of a physical embodiment of that model, if available, or optionally can be used as a specification of the physical implant to be generated/fabricated. By “anatomy implant model” (also interchangeably referred to herein as “implant model”, “anatomy model” or “anatomical model”) is meant a digital (i.e. formed of data) model of anatomy. This digital model can serve as a specification of a physical implant, and the specification can be used for fabrication of the physical implant and/or selection of an existing, e.g. commercial off-the-shelf, physical implant if such implant has already been fabricated.

Thus, in some embodiments, best-fit talus model(s) identified and selected from a database or library of models can be presented for potential use in selecting or fabricating a hardware implant of the desired characteristics. For instance, an anatomy implant model validated for a given patient can serve as the basis for identifying an existing implant or as a specification of an implant to be generated (e.g. fabricated). In this latter regard, an identified anatomy implant model can be loaded from a file, such as an .stl file, optionally manipulated, validated, and provided to fabrication equipment, such as a three-dimensional (3D) printer.

The search for best-fit implant model(s) may be based on anatomical surfaces of anatomy adjacent to the subject anatomy for replacement. For instance, the anatomical surfaces can be articular surface(s) of other anatomy with which the subject anatomy for replacement engages.

Various aspects are discussed and presented herein with reference to a talus (“ankle”) bone. The talus is one of a group of foot bones known as the tarsus. The tarsus forms the lower part of the ankle joint through its articulations. The talus bone can transmit the entire weight, or a substantial portion of the weight, of the body to the foot. The talus bone is extensively covered in cartilage and, together with the calcaneus (“heel bone”) and the navicular, forms the talocalcaneonavicular joint. The talus can include, as its three basic parts, (i) head, (ii) neck, and (iii) body portions. Example such articular surfaces in the context of a talus replacement are the calcaneal articular surface, the navicular articular surface, the tibial articular surface, and/or the fibular articular surface.

As discussed above, various injuries can be difficult to heal. The talus bone lacks a good blood supply for instance and, as such, individuals with a broken talus bone may not be able to walk for many months without crutches. One approach is to treat the injury with a talus implant. The insertion of a talus implant as a replacement of the patient's natural talus begins with an incision to expose the ankle joint, followed by movement or removal of the obstructing extensor hallucis longus tendon, the anterior tibial artery, and the extensor longus muscle. The talus can be held in place by the syndesmosis and anterior tibiofibular ligament, as well as the anterior talofibular ligament and the superficial deltoid ligament. The exposed, injured talus bone can be removed from the ankle joint and replaced with a talus implant inserted into the space formerly occupied by the patient's natural talus. However, since the talus bone interacts with other bones in the ankle, an accurate talus replacement and implant method/system is desired for accurate selection, optional creation, and implanting of a replacement talus in the patient. Existing methods use a ground up approach to talus replacement in which a custom talus is designed for each patient. This can be both time consuming and expensive. In accordance with some aspects described herein, machine learning is utilized to support the selection and generation of best-fit anatomical implants.

Although aspects described herein are presented with reference to a human talus, this is only by way of example. Skilled artisans will recognize that aspects presented herein can be used to inform implant selection/generation and other aspects for use with other anatomic features within the human body and/or other anatomies, and further that aspects described herein can apply to both total and partial anatomy replacement.

Thus, by way of example and not limitation, some aspects described herein utilize machine learning to support the selection and/or generation of best-fit implants, e.g., talus implants. Artificial intelligence, for instance in the form of one or more trained ML models, may be utilized to cognitively analyze certain anatomical parameters (e.g. articular surfaces) of a given patient and, from this analysis, select a best-fit anatomy implant model, e.g. a talus design. The selection in some examples is made from a library/database of pre-existing designs, for example digital three-dimensional (3D) models (or other form of specification) of taluses. In some examples, the analysis of anatomical parameters and selection of candidate best-fit model(s) could inform manipulations to a model that results in a new model for provision in the database. ML models employed herein can be self-learning and can improve through use. Additionally, parameters utilized to select an anatomical model, such as a talus model, for implant can be tuned via additional processing in order to select and generate a best-fit solution tailored to each individual patient.

Program code can determine properties, for instance measurements, distances, profiles, and other properties, of anatomical (e.g. articular) surfaces from images of a given patient anatomy and utilize these properties to select a best-fit anatomy implant model from a library of options and using machine learning. ML models utilized in accordance with aspects discussed herein can be of various types, for instance neural networks, including recurrent neural networks and/or convolutional neural networks. If certain data that would aid the program code in selecting a best-fit, pre-existing model is unavailable, aspects can generate a simulation for this missing data and utilize the simulation to select (and optionally fabricate) the implant. For example, if a portion of the talar anatomy is missing, that portion could be simulated for purposes of the selection/fabrication.

In an example embodiment, program code executing on one or more processors processes and analyzes data from images, for instance digital imaging and communication and medicine (DICOM) images of patient anatomy, generates a digital model of the anatomy, and automatically determines/selects, based on the digital model of the anatomy, an anatomy implant model from a library of implant models. This selected model, perhaps with manipulations applied to it, can serve as a candidate to replace the patient anatomy. The candidate can be subject to validation for the given patient. If validated, a corresponding physical implant can be selected and obtained if it is available, for instance available as an existing ‘off-the-shelf’ product. Alternatively, the physical implant can be fabricated according to specifications of the anatomy implant model and utilizing fabrication technique(s) such as 3D printing, as one example.

Thus, in some embodiments, software implementing AI can provide recommendation(s) as to appropriate anatomical model(s) for potential replacement of patient anatomy and therefore help identify implant solutions for selection, or optional fabrication, based on a given patient's anatomy. Present approaches to generating implants are highly manual, potentially imprecise, and customized, which can create significant work and add expense to the overall process. In contrast, aspects discussed herein can automate portions of an implant design/selection process based on training and application of machines learning models, and fit/design prototypes for custom implants for specific sets of conditions by identifying best existing designs from which to select/generate implants tailored to the patients.

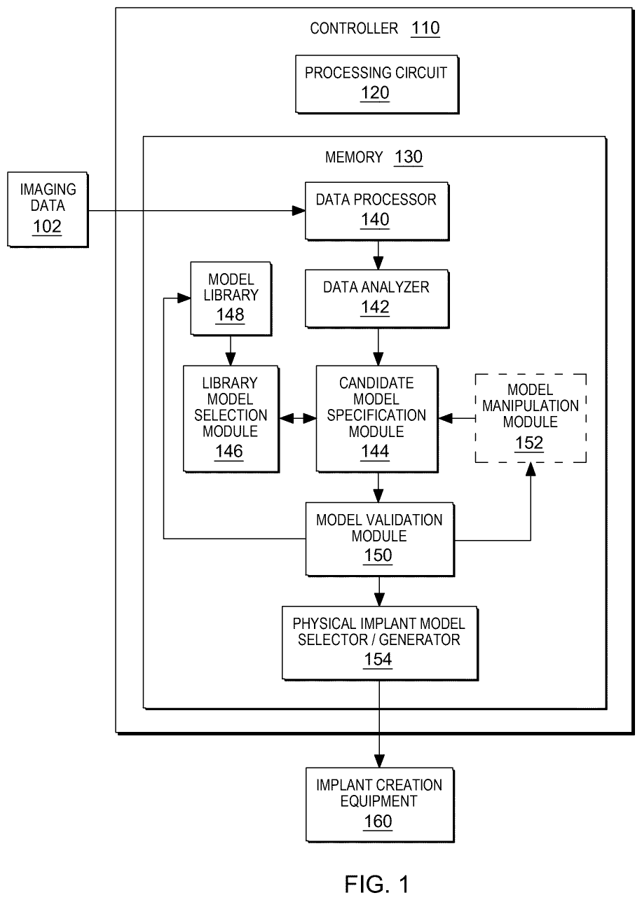

FIG. 1 illustrates an example environment to incorporate and use aspects described herein. The environment presents an example technical architecture and data flow illustrating certain general aspects that are explained in greater detail herein. The environment includes a controller 110, for instance a computer system, having a processing circuit 120 that includes one or more processors (as an example) and a memory 130. Memory 130 can include program code/instructions executable by the processing circuit to cause the controller 110 to perform functions, such as processes described herein. Program code of memory 130 can include various sets of code/instructions configured to perform specific activities. Shown in FIG. 1 are example such sets, referred to as modules or components. These are shown for conceptual illustration as separate elements within memory 130, however in other examples these modules can be further separated and/or combined. The specific delineation between modules shown in FIG. 1 is for illustrative purposes only.

A data processor module 140 obtains imaging data 102. In examples, the imaging data is obtained from an imaging device, such as medical imaging equipment, though in other examples it is obtained from a data store that stores imaging data. Imaging data can include images of anatomical regions and structures in any of various digital formats. One such format is based on the Digital Imaging and Communications in Medicine (DICOM) standard. Example imaging data is scan data (e.g. sets of images) generated from a computed tomography (CT) or other diagnostic imaging procedure.

The data processor module 140 processes the obtained imaging data 102 to perform any desired standardizing, cleaning, or initial processing thereof. A CT scan for example produces a stack of two-dimensional (2D) images that divide anatomy into very thin ‘slices’. A CT scan of a foot might produce 600 images for instance. The program code of the data processor module 140 can segment the imaging data, referring in this example to converting the 2D images into a 3D model. The data processor module 140 can therefore produce a 3D model from the input imaging data 102.

The anatomical region presented by the imaging data can include patient anatomy, including anatomy for potential replacement with an implant (referred to herein as “subject anatomy”) and other patient anatomy, at least a portion of which is adjacent to the subject anatomy.

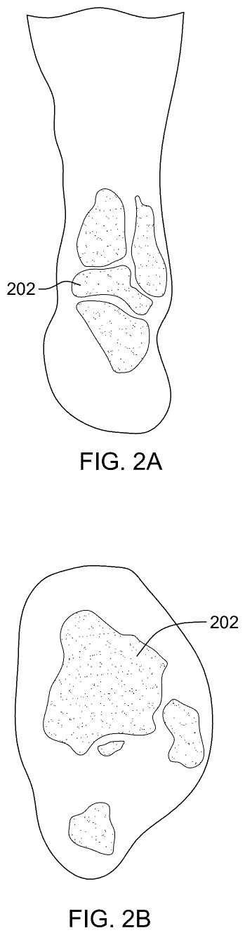

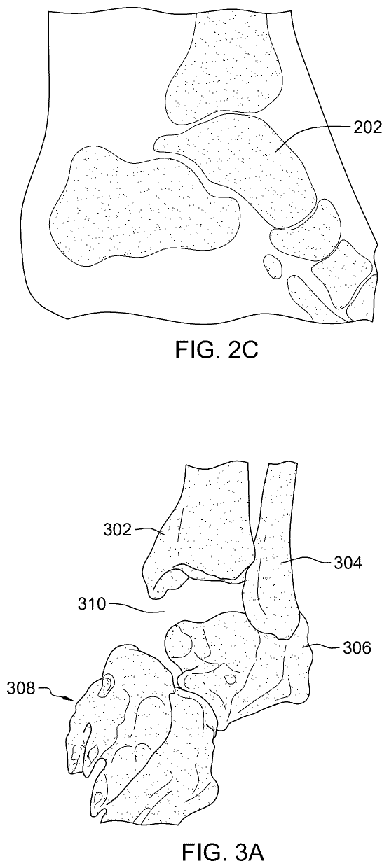

By way of specific example, FIGS. 2A-2C depict example loaded, segmented computed tomography (CT) images in accordance with aspects described herein. FIG. 2A presents a coronal planar image of a patient ankle, FIG. 2B presents an axial planar image of the patient ankle, and FIG. 2C presents a sagittal planar image of the patient ankle. The image data has been processed to segment different bone anatomy of the patient. The images shown present different bones.

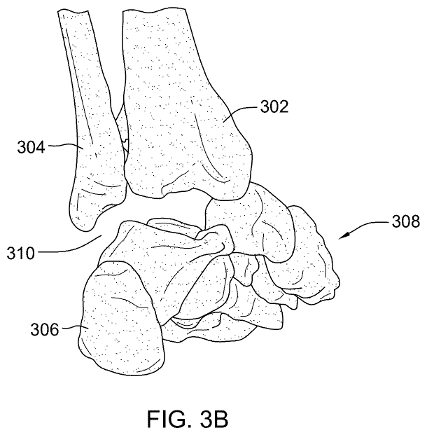

Given that program code is ultimately to be used in selecting/specifying a suitable implant for the subject anatomy (talus in these examples), the image data can be processed by data processor 140 and/or a data analyzer 142 to remove image data of the particular subject anatomy that the implant is to replace. The resulting model can be presented to a user for view. For instance, FIGS. 3A-3B present example anterior-lateral (FIG. 3A) and posterior-medial (FIG. 3B) views of a digital model of the patient's ankle region in which talus bone is omitted. Specifically, the tibia 302, fibula 304, calcaneus 306, and tarsal/metatarsal bones 308 are depicted surrounding the space 310 in which the talus sits but has been removed programmatically.

Processed data from data processor 140 is provided to data analyzer 142 for analysis thereof. The data analyzer 142 is used in the identification of properties of anatomical surfaces adjacent to the subject anatomy. In examples, the anatomical surfaces are articular surfaces, i.e. surfaces of anatomy with which the patient's talus (in this example) engages and therefore with which an appropriate talus implant is expected to make contact when provided within the patient. Thus, as part of the data analysis by analyzer 142, articular surface(s) of patient surrounding anatomy are identified. In some examples, the articular surfaces are identified/specified in whole or in part manually by a user (a doctor, an engineer specializing in virtual surgical planning, etc.) who uses a computer system, display, and input devices to identify boundaries and other properties of these articular surfaces. Additionally or alternatively, AI based on machine learning may be able to perform this identification by way of a model trained to take image data as input and identify articular surfaces of adjacent anatomy presented in that image data. For instance, program code of data analyzer 142 could apply machine learning algorithm(s) utilizing a neural network to detect and determine properties of articular surfaces presented in the images. In the context of examples presented herein, the articular surfaces are other bone surfaces of at least some bones with which the talus makes contact, e.g. surfaces of the navicular, calcaneus, fibula and/or tibia bones.

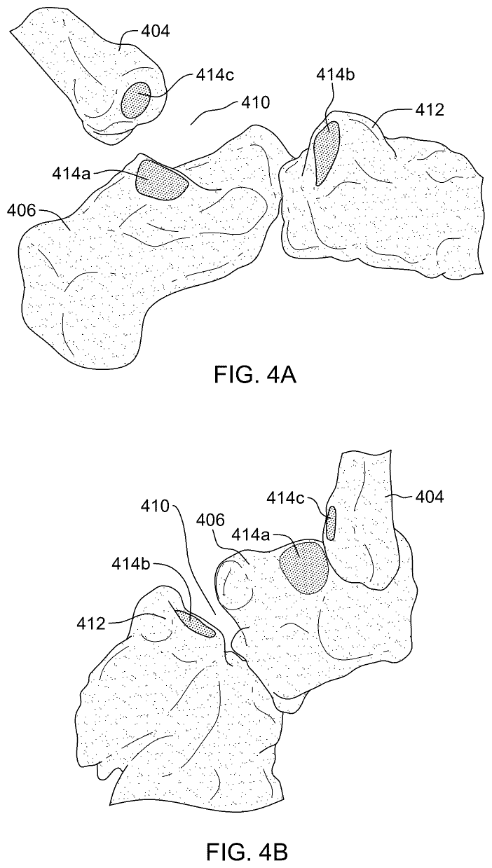

FIGS. 4A-4B depict the example patient bone model of FIGS. 3A-3B with the aforementioned three articular surfaces highlighted. The model with highlighted surfaces can be rendered for user view. In FIGS. 4A-4B, the fibula 404, calcaneus 406 and navicular 412 bones are shown in position relative to each other, with space 410 for a talus implant. 414a denotes the calcaneal articular surface between the calcaneus and talus, i.e. the portion of the surface of the calcaneus with which the talus physically touches/engages and articulates, 414b denotes the navicular articular surface between the navicular and talus, i.e. the portion of the surface of the navicular with which the talus physically touches/engages, and 414c denotes the fibular articular surface between the fibula and talus, i.e. the portion of the bottom surface of the fibula with which the talus physically touches/engages. The software can determine and/or ascertain based on user input various properties (such as size, shape, perimeter, area, surface contour, etc.) of these surfaces to help inform model selection/generation processing as described.

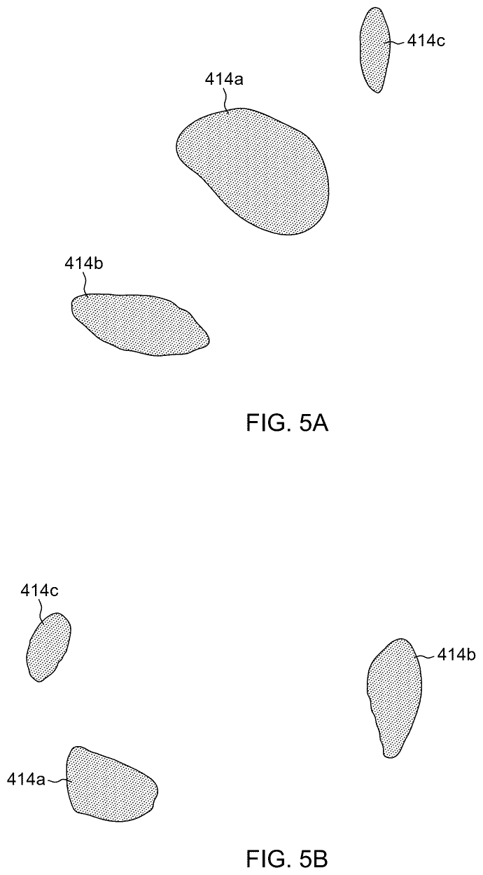

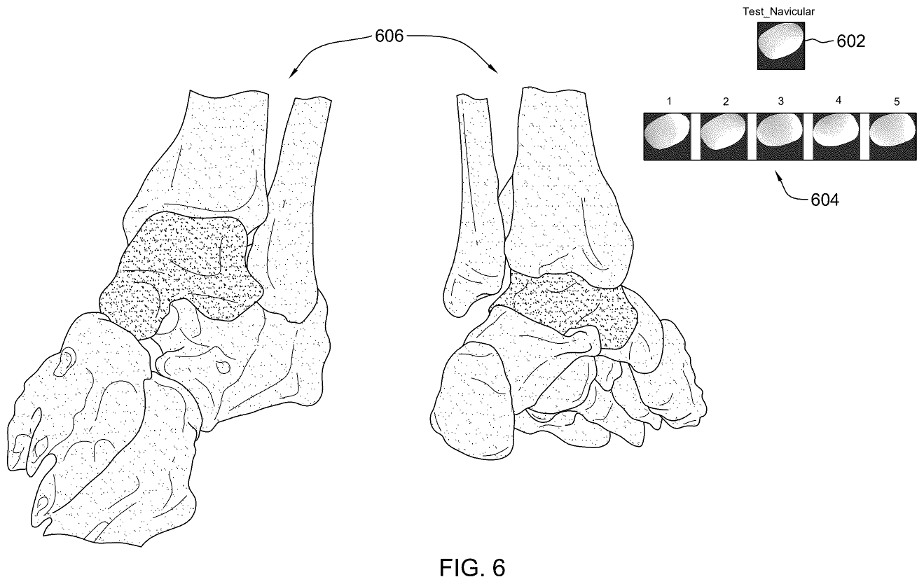

FIGS. 5A-5B depict properties (such as dimensions, perimeter, and position relative to each other) of the articular surfaces of FIGS. 4A-4B, with the surfaces 414a, 414b, 414c isolated from other patient anatomy, in accordance with aspects described herein. Aspects can present this view on a display to a user if desired.

In some situations, the anatomical structure to identify an articular surface is not present. In these cases, the program code could infer missing portions of the anatomy from image data 102 that is available. For example, if the program code is to determine an implant to be used on a left side of a patient and a desired articular surface is not present from the image data 102, the program code could infer the missing surface based on the available data.

Referring back to FIG. 1, with the articular surface(s) identified by way of the data analyzer 142, processing of the controller can progress to the candidate model specification module 144. In some aspects, the candidate model selection module 144 can use the articular surfaces identified from 142 to inform limits (as distances, perimeters, and other parameters) for selection of candidate implant models to fill the implant space. Module 144 can leverage model selection module 146 to selects/identify best-fit anatomy implant model(s) from model library 148, which is a library/database of anatomy implant models. The models in the model library 148 could include models of actual human taluses and/or models that are at least partially specified/manipulated by users, as described below. In examples, the model selection module 146 uses ML model(s) as discussed herein to find closest point(s) in an n-dimensional latent space to select best-fit implant models existing in the library 148.

Optionally, as described herein, manipulations can be applied to a selected implant model, with such manipulations being facilitated by model manipulation module 152.

In any case, the candidate model specification module is used to specify a candidate implant model to undergo validation. The validation determines whether the candidate is an appropriate specification to use for the given patient of which the imaging data 102 was taken. As an example, the user selects as a candidate a best-fit model identified by 146 and this selected candidate is provided for validation. Optionally, selected best-fit models returned from 146 can be presented in an interface for view by a user and the user can select a best-fit such model as an initial selected implant model. Alternatively, a best-fit model could be automatically selected (e.g. by 146 or 144) and optionally confirmed as such by the user. Feedback in the form of user selection or confirmation as to a best-fit design for a given input can be utilized by program code to update the machine learning algorithms utilized to select best-fit designs from the model library, if desired.

With the candidate model having been specified, the candidate model is provided to model validation module 150. The validation module 150 receives the candidate model and attempts to validate it. Validation could be manual, automatic, or a combination of the two. In a particular example, the user performs at least some of the validation by identifying digitally, e.g. using the controller, whether the implant fit is appropriate or, alternatively, identifies that some adjustments are needed. Additionally or alternatively, the validation is in some embodiments at least partially automated (e.g. using a machine learning model) to validate whether a selected best-fit implant model (possibly with manipulations applied) is an acceptable fit. The validation can check whether the physical implant specified by the candidate implant model passes for surgical implantation within the patient. Validation could involve any one or more desired validation processes, tests, checks, or the like, performed with respect to the selected implant model or collection of candidates for selection.

In one example, validation includes an assessment—wholly manual, wholly automatic, or a combination—as to the distances between a selected implant and various patient anatomy when the implant is in operative position. It may be desired, for example, to examine based on surface mappings an average gap size between a surface of the implant and corresponding articular surface of patient anatomy with which that implant surface interfaces. In examples, the desired gap size may be between 0 and 3 millimeters, and more particularly between 1 and 2 millimeters. In general, spatial and/or volumetric analysis may be useful in determining the fit of the implant with the surrounding anatomy. As another example, validation might utilize a motion simulation of the implant model positioned within the patient to simulate and assess how fit is informed by patient movement. A gait cycle or other motion simulation, for instance, can simulate implant movement, shifting, and the like based on simulated user movement. This can inform appropriateness of the selected implant for the particular patient.

There are some situations, particularly those in which a patient had an infection or other event that has resulted in removal of the anatomy prior to formulating a replacement plan, where surrounding anatomy (such as soft tissue) changes such that the space in which the implant is to fit has different characteristics by the time implant selection is considered. For instance, in the case that soft tissue begins to populate the void left by the removed anatomy, distraction of the joint may be desired, albeit not to the extent that a talus implant matching the size of the patient's natural anatomy is appropriate. The talus implant may be selected such that it is smaller than the original talus, larger than the current space that it is to occupy, and accomplishes the desired level of joint distraction. Validation in this context could check whether the implant is expected to accomplish the desired amount of distraction.

Validation could incorporate input from a collection of doctors or other health care professionals. For instance, validation could be based on responses from the collection after presenting selected implant model(s) in conjunction with specific patient anatomy/anatomies. In an embodiment, the collection of doctors could be polled or the like to indicate proper fits of implant-anatomy pairs. In a specific example, a collection (in the form of a grid, for instance) of implant models are presented as candidates for one or more corresponding patient anatomies, and the doctors are prompted to indicate which indicate proper fits. Responses can be collected as a form of crowdsourced input as to proper implant fit. Proposed implant-anatomy combinations could be presented in any desired manner, including cross-sectional or other static views and/or as motion simulation(s) discussed above. In some examples, responses are used to train machine learning model(s) to validate selected implant models for given anatomy. Additionally or alternatively, the responses can inform statistical ‘rules’ (acceptable ranges, averages, etc.) useful for implant validation.

If the candidate model is validated, a physical implant model selector/generator 154 is used to either select a make/model of an existing physical implant (e.g. commercial off-the-shelf) if one can be identified from the specification, or initiating generation/fabrication of the model by passing a specification of the implant to implant creation equipment 160 to fabricate the implant. This specification can direct the creation of the implant. For instance, in some examples the specification in digital form can be provided to implant creation equipment 160, which may be, as an example, a 3D printer or other additive manufacturing equipment or supporting controller thereof. Implant creation equipment 160 can be controlled by program code of the controller and/or other computer system(s) to automatically create the implant.

If the candidate is not validated, then processing can proceed back to candidate model specification module 144 via manipulation module 152. The manipulation module 152 enables optional manipulation to a model as part of candidate model specification. For instance, a user can optionally manipulate a candidate model, e.g. a model selected from the model library and/or a candidate model that fails validation. A manipulation may be made in situations where the user and/or a process identifies that a particular candidate model is not sufficient for the given patient. As examples, the user or a process might observe that the candidate model is intolerably (e.g. exceeding one or more thresholds) small for the space to be occupied, or the areas on the implant contacting the articular surfaces of surrounding anatomy are not satisfactorily in-alignment with those surfaces. Additionally or alternatively, a user might want to drive anatomical changes such as joint distraction by way of implant selection. Accordingly, the manipulation module can be used by a user to manually adjust sizing, shape, and other properties of the implant model to better fit the model to the patient. Optionally, such adjustments could be made automatically in some embodiments.

In the alternative, or in addition to, manipulation(s) to the implant model, the manipulation module 152 could be used to manipulate the size, position, distances between features, and/or other properties of the patient anatomy as reflected by the input data 102 to thereby modify the patient anatomy upon which model selection and/or validation is based. This includes possible changes to properties (size, position, distances between features, etc.) of the identified articular surfaces from 142. Modifying the patient anatomy might result in the identification of a better candidate implant model, whether it is one that was presented to validation module 150 as a candidate implant and failed to validate at the validation module 150 or one that is identified after an updated search of the model library 148 via selection module 146. Additionally, a candidate implant that fails validation initially might pass without further manipulations to the implant model itself if appropriate patient anatomy manipulations are applied. Some manipulations of patient anatomy may be acceptable in the right circumstances since patient anatomy could naturally adapt to an implant.

In some examples, the library model selection module 146 selects a plurality of implant models that are the ‘best-fitting’ models from the library for the given patient anatomy. The selection module 146 might rank these multiple best-fit models by confidence or other indication of how well the ML model used by the selection module 146 predicts them to. The ‘best’ such model of the ranking can be selected automatically or manually by a user as an initial selected implant model. This initial selected model can be sent as-is to the validation module 150 for validation, effectively making the selected implant model the initial candidate implant model to attempt to validate and, if this candidate is rejected, the processing could return to candidate model specification module 144 for selection of a next best fit model of the ranking as a next candidate and/or manipulation of either the candidate implant model, the patient anatomy, or both. Automatic or user manipulation via 152 may or may not be performed at each iteration, and either or both may be optional. A specific example process iterates back to 144 without any modification to the candidate and selects, as a next candidate to try, the next best ranked model in the plurality that was selected by the library model selection module 146. However, as noted above, the user could apply manipulations to a selected/candidate implant model on any given iteration, or when a next model is selected. The user can have criteria to understand whether manipulation(s) to the anatomy or to a candidate model that failed validation may result in successful validation, and whether, instead, it would be better to select as a different model from the library as a next model with witch to work. AI could be used in this decision-making as well, for instance to identify situations when working with a current model and applying manipulation(s) to it and/or the anatomy may be better than switching to a next selected model selected by 146, e.g. from the library 148.

It is seen that the candidate model specification module 144 is used to specify candidate models for validation. The candidates for validation may be as they are provided in library 148 and/or may be manipulated automatically or by a user and then sent for validation. In situations where a candidate model that has passed validation either (i) does not exist in the library 148 or (ii) exists in the library but has not previously been validated for anatomy corresponding to that patient anatomy with which it was just validated, then this results in potentially useful training feedback for the system. For instance, the specification of a validated implant model not already in the library 148 can be added (151) to the library. While a manipulated model may not represent a corresponding natural anatomy from a patient, it was nevertheless found valid and verified for the given patient anatomy, and therefore can be added as a legitimate implant model to the library 148. Then, or if the implant model was previously known in the library but not in connection with anatomy corresponding to that patient anatomy or manipulation(s) applied thereto, this can be provided as further training data for training the ML model used by the library model selection module 146 to identify best fit implant models for given input patient anatomy (e.g. articular surface properties).

In some examples, the initial candidate model is selected as the best-fit from the library and the user or system might manipulate the model as an initial adjustment prior to attempting validation. In any case, processing in accordance with FIG. 1 can iterate through candidate(s) and/or manipulations until a model is identified that is validated, after which a physical implant is obtained/fabricated and used. In this manner, a process can iterate until an appropriate and validated candidate implant model has been specified. This process might include manipulation(s) to patient anatomy reflected by the input data and/or to implant model(s) put for validation. Manipulations to an implant model may result in the definition of a new implant model not seen in the library. In this case, and assuming the new model is validated for the given patient anatomy, the new model can be saved to the library and made available for later selection. Meanwhile, it can be provided as a further training example together with the properties of the corresponding articular surfaces with which the model was validated.

In examples, a graphical user interface (GUI) is provided through which a user invokes processing described herein, including selection and specification of implant model(s) based on one or more best-fit options selected by program code (e.g. library model selection module 146) having applied one or more trained machine learning algorithms. The GUI could enable a user to upload digital file(s) comprising image data (e.g., FIG. 1, 102) and specify certain search criteria and/or a search method(s) to use. The GUI can invoke processing such as that described herein to select and present to the user the best-fit model(s). Based on these search results, the user can select a given result as a selected implant model for provision to the candidate model specification module 144 and/or a next selected implant model could be automatically selected for provision to the candidate model specification module 144. The user may have the option to perform manipulations to the model and/or patient anatomy (including, for instance, the articular surfaces).

The program code can search based on one or more of the articular surfaces and display the best-fit model(s) themselves from which a user can select a next implant model for provision to 144 as a potential candidate. In other examples, the program code obtains the image data and automatically searches/selects a model.

FIG. 6 depicts an example interface presented by such a GUI for view and selection of a best-fit implant model in accordance with aspects described herein. Element 602 presents a 2D rendering of the navicular articular surface of the patient's existing anatomy, i.e. the portion of the surface of the patient's navicular bone that is expected to physically engage with the talus implant. Properties of this surface can be an input to a ML model of, or leveraged by, the library model selection module 146. The library model selection module 146 determines a top one or more (5 in this example) best-fit models, denoted by 604 which present 2D renderings of the navicular-engaging surfaces of the top 5 best-fit models.

2D renderings of the surfaces are shown in FIG. 6 as just one example of a manner in which to present the input and results of library model selection. In this regard, some neural networks and other ML models for AI may not perform best when using 3D model data. In these cases, the 3D surfaces representing the articular surfaces to be used as input to the model can be provided as 2D projections. 602 of FIG. 6 is a 2D projection of the 3D navicular articular surface of the patient. Different colors, shades, numerical values, or other data can be used to represent the third, ‘depth’ dimension with each point corresponding to a distance between the point on the articular surface and a flat plane behind the surface. In the examples of 602 and 604 in FIG. 6, points ‘closest’ to the 2D plane are shown in darkest shade and points farthest from the 2D plane shown in lightest shade. Representing the 3D articular surface as 2D data enables AI to more efficiently work with the properties of the articular surface. This 2D rendering may be optional, since other already-existing or to-be-developed AI may be able to work with the 3D data as-is.

As noted, 604 presents the top 5 best-fit models, represented by their surfaces that will engage with the patient's navicular bone. The user can select one of these results and the GUI can display the selected implant together with the surrounding anatomy. 606 of FIG. 6 depicts anterior-lateral and posterior-medial views of the patient ankle incorporating a selected implant model 608.

Accordingly, program code of FIG. 1 can ascertain articular surfaces of a patient anatomy to engage with an implant. The library model selection module 146 can determine best-fit model(s) for the implant selected from a library based on properties of these articular surface(s). In order to select best-fit model(s) (e.g., those with surfaces expected to most appropriately engage with the identified articular surfaces of the patient anatomy) the library model selection module 146 can utilize classifiers, including image classifier(s), such as Artificial Neural Network(s) (ANN), convolutional neural network(s) (CNN) (such as Mask-RCNNs), Autoencoder Neural Network(s) (AE), Deep Convolutional Network(s) (DCN), and/or other image classifiers and/or segmentation models, and combinations thereof. In examples that utilize a CNN, the CNN can be configured utilizing an AI instruction set (e.g., native AI instruction set or appropriate AI instruction set). In certain examples herein, the classifiers utilized are deep learning models. Nodes and connections of a deep learning model can be trained and retrained without redesigning their number, arrangement, interface with image inputs, etc. In some examples, these nodes collectively form a neural network. In certain embodiments, the nodes of the classifier may not have a layered structured. To configure the neural network, program code can connect layers of the network, define skip connections for the network layers, set coefficients (e.g., convolutional coefficients) to trained values, set a filter length, and determine a patch size (e.g., an n×n pixel area of the image, where n can be 3, 4, etc.), as examples.

In some embodiments, program code utilizes a neural network to select a model from the model library in real-time such that a user providing inputs into the GUI can obtain results near-immediately. In some examples, program code includes a pre-trained CNN configured to classify aspects of the images obtained by the program code. In some examples, the CNN can be MobileNet or MobileNet2 and available in libraries such as Keras, TensorFlow, and/or other types of libraries. Classifiers(s) of a model selection module can be generated, by program code in embodiments, from a pre-trained CNN that, for instance, deletes, modifies, and/or replaces layer(s) of neurons (e.g., input layer, output layer).

In accordance with aspects described herein, program code, such as that of a library model selection module 146, can implement a ML model that is trained to learn the key features of anatomy with a focus on the articular surfaces, and identify one or more best-fit designs from the model library. In some examples, program code includes a deep neural network and it is this deep neural network that is trained to learn the features of the designs in the model library 148, including the corresponding articular surfaces with which they pair, for proper classification of input.

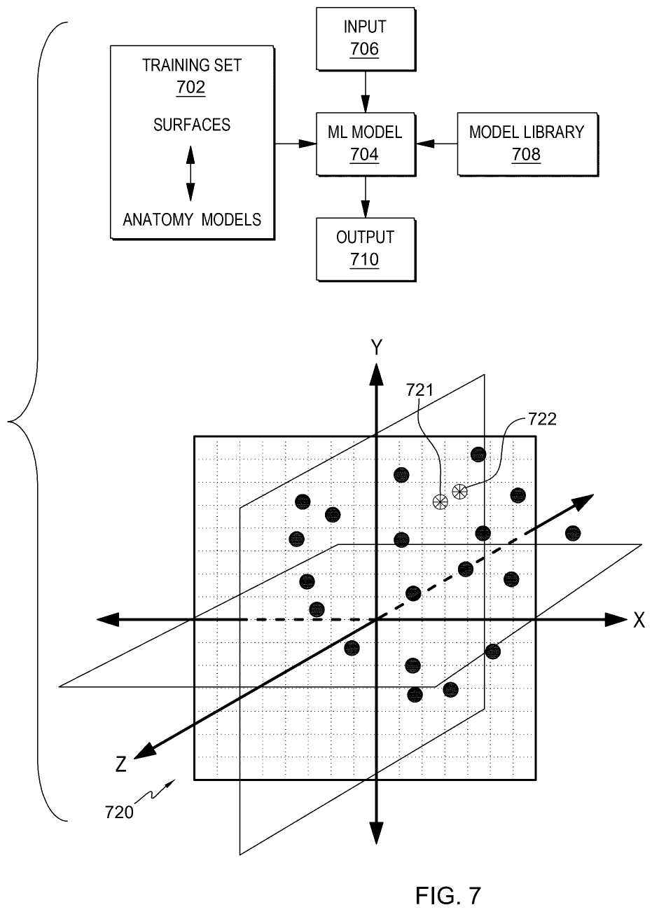

FIG. 7 depicts a conceptual illustration of machine learning model training and use in accordance with aspects described herein. Training dataset 702 is used to train ML model 704. The training dataset 702 can include models of example adjacent anatomy (e.g. articular surfaces) and/or their properties, together with correlated anatomical implant models that ‘fit’ with that adjacent anatomy. Thus, using the examples described herein, the training dataset can include models of taluses (which may be models of actual human taluses) and models (e.g. 3D and/or 2D) of the articular surfaces with which those taluses were paired. Properties of the articular surfaces and the taluses pairing with them can be determined by preprocessing of actual patient image data, for instance.

The ML model 704 can be any type of ML model, a specific example of which is an autoencoder, which is a type of neural network. Training the ML model 704 teaches the ML model 704 to identify talus models that are compatible with given input articular surface(s). Such talus models can correspond to existing implants or implants to be fabricated. The ML model 704 may be trained with any volume of training data, though it is generally the case that a greater number of training examples will produce greater accuracy than would a lesser number of training examples. In a particular example, the ML model 704 may be trained to produce acceptable results based on approximately 200 training cases (articular surface-bone model pairs).

In general, the training teaches the ML model 704 to identify the most relevant patterns in the data such that when properties of articular surfaces adjacent a given subject anatomy for replacement are input to the model, the model returns an appropriate implant model for the anatomical replacement. Dimensional reduction to a ‘low dimensional space’ takes the most representative patterns in the anatomy as features to look for when provided an input 706. In examples, input data can be 3D models of anatomy that are converted to 2D data (e.g. projections as described above), then fed as input 706 to ML model 704. Model 704 is trained to identify implant models from model library 708 that fit with the given input. The model library 708 can include implant models corresponding to the examples in the training dataset, and/or other implant models. The ML model 704 will provide zero or more selected model(s) as output 710. If multiple models of ‘best-fit’ implants for the given input are identified, they may be ranked or unranked.

The models in the model library 708 and any ‘hits’ based on an input can be represented by 720, an ‘n-dimensional latent space’. The example of 720 shows three dimensions, but in many practical examples the latent space is n-dimensional where n may be greater than 3. Each point in the latent space represents a possible output label, e.g. an implant model in these examples. Thus, each specific talus model may be represented as a unique point in the latent space. In this example, two dots 721, 722 represent two ‘hits’-best-fit implant models based on hypothetical input surface(s) that were put into the trained ML model.

In some embodiments, the ML model may be provided as a 3D generative adversarial network (GAN) structure. The GAN approach trains a discriminator to discriminate between real/actual samples and fake/generated samples from a generator. Meanwhile, the generator is being trained to generate better samples. Once the generator is sufficiently trained, it can be used as the classifier/ML model for input anatomical properties. In the context of aspects described herein, a generator can thereby be trained to generate implant models (e.g. total talus models) from scratch providing a manner of selecting an implant model in the alternative or in attrition to selecting best-fit model(s) from an existing library. The generator effectively learns from the patterns present in the inputs with the goal being for the generator to generate an anatomy (e.g. talus) that a human would have naturally generated for the given anatomy. The result produced by the generator can be a point cloud. The point cloud can be processed using various filtering and interpolation techniques to produce closed surfaces and ultimately a continuous volume representative of a full anatomical structure, such as a talus.

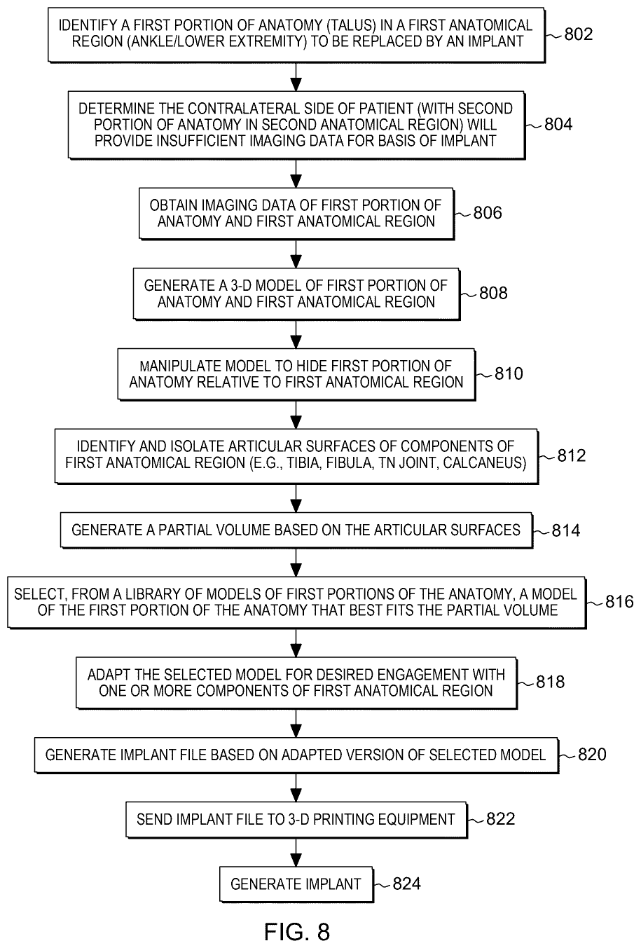

FIG. 8 depicts an example process flow for artificial intelligence-based implant selection, in accordance with aspects described herein. The process can be performed in whole or part by one or more computer systems executing program code. The process identifies (802) a first portion of anatomy (e.g. talus) in a first anatomical region (e.g. ankle/lower extremity) to be replaced by an implant. Optionally the process can make a determination (804) (based on an inquiry and other processing, for instance) that a contralateral side of the patient (e.g. having a second portion of anatomy in a second anatomical region) does not alone provide sufficient imaging data to form a basis of the desired implant to replace the first portion of anatomy. In this case, the process therefore obtains (806) imaging data of the first portion of anatomy and first anatomical region, and generates (808) a 3D model incorporating the first portion of anatomy and first anatomical region. The process manipulates (810) the 3D model to hide the first portion of anatomy relative to first anatomical region, and then identifies and isolates (812) articular surfaces of components (e.g., tibia, fibula, talo-navicular joint, calcaneus) of the first anatomical region with which the first portion of the anatomy interact. The process can generate (814) a partial volume based on the articular surfaces, i.e. the articular surfaces inform a portion of the periphery of the volume/implant to occupy the space. The partial volume represents at least some limits/dimensional extremes of the implant. The process then selects (816), from a library of models, one or more implant model(s) of the first portion of the anatomy that best-fit(s) the partial volume. The process can adapt (818) one or more such selected model(s) for desired engagement with one or more components of the first anatomical region, and these components may be the articular surfaces and/or other components of the patient anatomy. Eventually, a model specification may be validated at which point the process generates (820) an implant specification file based on the validated model specification and sends (822) the implant specification file to 3D printer or other implant fabrication equipment, which implant fabrication equipment generates (824) the implant. As an alternative to 820, 822 and 824, an existing physical implant to which the validated model specification corresponds can be selected and obtained. In either case, once the physical implant is obtained then it can be surgically implanted within the patient.

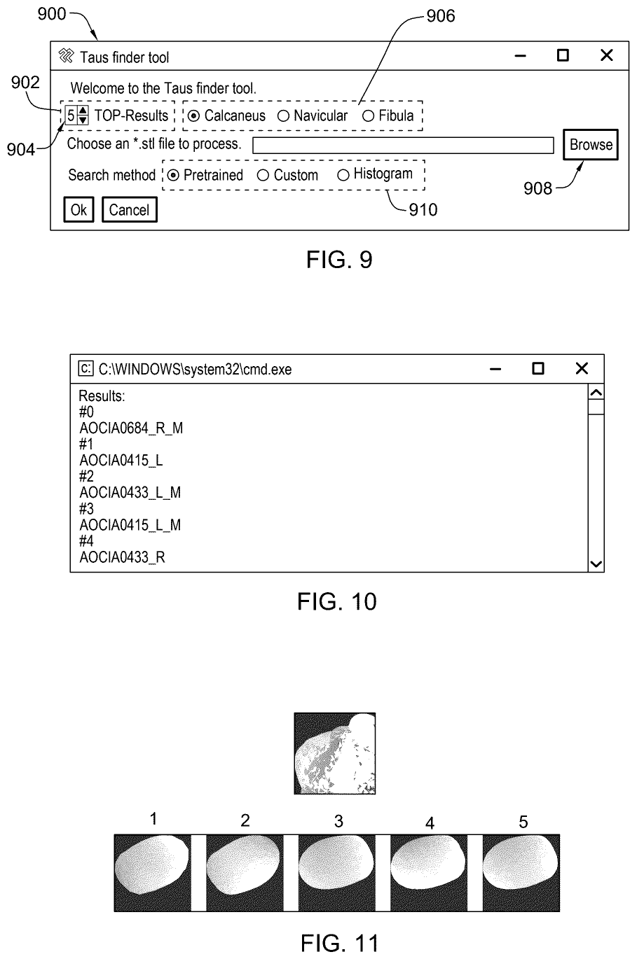

FIG. 9 depicts an example graphical user interface (GUI) of software for performing aspects described herein. The GUI can be utilized to identify and present best-fit model(s) to the user for selection, e.g. as part of at least library model selection module 146 of FIG. 1. Interface 900 is presented on load of the software. A TOP-RESULTS portion 902 includes an option 904 for the user to select how many outputs (e.g. best-fit models) will be selected and provided by the ML model as potential implant models for use. Option 904 can be configured with a default, for instance 5 as shown. The option 904 is implemented in this example as a number picker with up-down selectors.

Interface 900 also includes an anatomy type selection portion 906, which enables the user to select which anatomical surfaces (articular surfaces here) the software is to use in its comparison of the models in the library to the anatomy model loaded by the file input portion 908. In this example, the anatomy type selection portion 906 is implemented as three radio buttons corresponding to calcaneal, navicular, or fibular articular surfaces and the user is to select one of the three. In embodiments in which multiple articular surfaces may be considered in the selection of a best-fit model, the surface type selection portion 906 may be implemented using a different interface element type to enable the user to indicate more than one articular surface. In the example of FIG. 9, the user has selected (and/or the program uses by default) the calcaneal articular surface.

File input portion 908 enables the user to provide a file to process. In this example, the software takes an .stl file of the anatomy. The user selects the Browse button to browse a file system to the appropriate .stl file containing the model of the patient anatomy. The file may be opened/loaded upon selection by the user.

The user also selects a search method in interface portion 910, implemented as three radio buttons corresponding to pretrained, custom, or histogram search methods. In the pretrained search method, the search leverages an ML model (for instance a convolutional neural network) trained with a dataset and then a last layer is added with classes/labels corresponding to available implant models of the database. The training could be based on any desired anatomy, specific to anatomical regions (e.g. shoulder, ankle, knee, etc.) or more general (full body CT or scans for instance). A custom search method may train the ML model in a fashion more customized to a particular condition, deformity, or the like (i.e. less generic than the pretrained method). A histogram search method may instead be based in statistics and statistical modeling that compares selected anatomy to ‘normal’ (as informed by averages) anatomy-measurements, curvatures, etc. that are stored in a database of normal anatomy. This could inform a degree of correlation between actual patient anatomy and the average/normal anatomy and identify desired properties for patient anatomy and therefore implant characteristics to inform appropriate implant(s) selection.

Once these parameters are specified, the user selects the OK button to cause the system to initiate processing and indicate best-fit result(s). In embodiments, indications of the results can be output to a command line interface as shown in FIG. 10, which identifies, in this example, 5 results (#0-#4) by name or other identifier of the implant model. Additionally or alternatively, the interface presents a 2D graphical rendering of an input articular surface and 2D renderings of the identified articular surface profiles corresponding to 5 best-fit results, as shown in FIG. 11.

Thus, based on the search results selected by the program code after conducting the search, the software can display one or more best-fit results. In some examples, a user selects a given result. Any one or more of these results can be used in conjunction with other aspects described herein, for instance aspects of FIG. 1 such as the candidate model specification module 144. In embodiments in which searches based on multiple articular surfaces are conducted, different sets of best-fit results, each corresponding to one of the articular surfaces, can be provided. Additionally or alternatively, the software can display full models themselves from which a user can select.

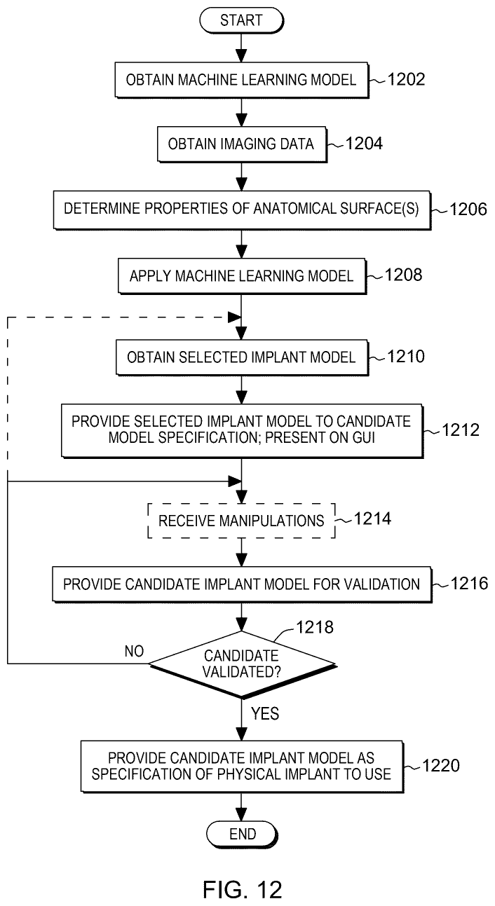

FIG. 12 depicts an example process for implant selection in accordance with aspects described herein. In some examples, the process is performed by one or more computer systems, such as those described herein.

The process obtains (1202) a machine learning (ML) model that has been trained to select, based on properties of anatomical surfaces of anatomy that is adjacent to subject anatomy to be partially or totally replaced, anatomy implant models of physical implants for partial or total replacement of the subject anatomy. The method also obtains (1204) imaging data of an anatomical region of a specific patient. The anatomical region includes a subject anatomy of the patient and other anatomy of the patient, where the other anatomy is adjacent to the subject anatomy of the patient. In examples, the subject anatomy is a talus of the patient and the other anatomy are the navicular, calcaneus, tibia and/or fibula.

The process proceeds by determining (1206), from the imaging data, properties of at least one anatomical surface of the other anatomy. The at least one anatomical surface is at a respective at least one interface between the other anatomy and the subject anatomy of the patient. In examples, the anatomical surfaces are articular surfaces. The anatomical surface(s) can include articular surface(s) with which the physical implant is to engage based on being surgically implanted at least partially within the patient. In a particular example, the anatomical region comprises a patient ankle, the subject anatomy comprises a talus, and the at least one anatomical surface comprises at least one articular surface of at least one bone adjacent to the talus, such as such as navicular, calcaneal, tibial and/or fibular articular surfaces of the respective bones around the patient's talus.

A user might manually identify the surfaces and/or a process, perhaps applying a machine learning model, might automatically identify the surfaces. Additionally, the user might optionally make adjustments to identified surfaces. Thus, determining (1206) the properties of the at least one anatomical surface can be based on (i) manual indication, by a user, of the at least one anatomical surface provided based on user input to a graphical user interface displaying a model that includes at least the other anatomy of the patient, and/or (ii) automated analysis of the imaging data to ascertain the at least one anatomical surface.

In some embodiments, preprocessing is performed to convert 3D data to 2D data. Thus, the obtained imaging data can include three-dimensional digital model data representing the anatomical region of the patient, and the determining (1206) the properties of at least one anatomical surface of the other anatomy can include processing the imaging data to present the at least one anatomical surface as at least one digital three-dimensional surface, and converting the at least one digital three-dimensional surface to at least one two-dimensional projection, where the determined properties of the at least one anatomical surface are determined from the at least one two-dimensional projection.

Continuing with FIG. 12, the process applies (1208) the ML model, using the determined properties of the at least one anatomical surface, and obtains (1210), based on the applying, a selected implant model. The selected implant model is selected by the ML model as a specification of a physical implant for potential surgical implantation at least partially within the patient as a partial or total replacement of the subject anatomy of the patient.

The ML model can be trained using samples from a library of implant models. The training can train the ML model to select the anatomy implant models from the library of implant models, and therefore the selected implant model can be selected by the ML model from the library of implant models.

Alternatively, the ML model can include a trained generator of a generative adversarial network, wherein the generator is trained using samples from a library of implant models, and is trained to generate implant models. In this case, the selected implant model can include an implant model generated by the generator and selected by the generator for output as the selected implant model.

This selected implant model may or may not be appropriate for this patient. Accordingly, the process proceeds by providing (1212) the selected implant model to a candidate model specification module for specification of a candidate model for validation. The candidate model specification module can handle the selected model for, e.g., possible manipulation and/or other tasks. In this regard, the selected implant model may or may not be what is initially presented for validation. Additionally, the selected implant model can be presented to a user on a graphical user interface. Optionally, the method can preprocess the imaging data to produce a three-dimensional digital model of the anatomical region of the patient with the subject anatomy omitted therefrom and present this for display to the user.

The process also includes optionally receiving (1214) manipulations. The user might desire to manipulate the selected model at this point (or after a failed validation), which would result in a different model specification and therefore different specification of the physical implant that the model informs. Thus, in one aspect, receiving (1214) manipulations can include receiving manipulations to the selected implant model, where the manipulations change the selected implant model and produce a candidate implant model for validation. Based on such manipulations, the candidate implant model specifies a physical implant with a different one or more physical properties in comparison to the physical implant specified by the selected implant model that was initially selected and provided prior to the user manipulations thereto. As examples, the manipulations to the selected implant model could include manipulation(s) specified by the user and/or manipulation(s) determined automatically by artificial intelligence and optionally automatically applied.

Additionally or alternatively, the user might desire to impart manipulations on the representation of patient anatomy (e.g. surfaces). Thus, receiving (1214) manipulations can include receiving manipulations to the properties of the at least one anatomical surface of the other anatomy. For instance, the user might change the shape or other properties of the surfaces and/or the distances between them. If the user shifts a bone or other anatomy, this can change the position of the anatomical surface of that bone. The manipulations to the properties of the at least one anatomical surface can include manipulation(s) specified by the user and/or manipulation(s) determined automatically by artificial intelligence and optionally automatically applied.

Regardless whether any manipulations were applied, at this point a candidate implant model has been obtained, either as obtained and provided by 1210/1212, or after manipulation thereof. The process proceeds by providing (1216) the candidate implant model to a validation module for validation. The validation determines whether the physical implant specified by the candidate implant model passes for surgical implantation within the patient. The validation will be performed against the candidate that it is presented, and therefore, in the case where manipulations were made, with consideration of such manipulations. Thus, the validation to determine whether the physical implant specified by the candidate implant model passes for surgical implantation within the patient is based at least in part on any manipulated properties of the anatomical surface(s) and/or made to the implant model itself.

The process determines at 1218 whether the candidate implant model passes validation, i.e. whether the physical implant specified by the candidate implant model passes for surgical implantation within the patient. If not (1218, N), the process can return to 1214 where, using the candidate implant selection module, manipulation(s) to the candidate that just failed validation and/or to patient anatomy can again potentially be received. That informs a next candidate, which may a modified or unmodified version of the initial candidate that failed, and this next candidate is provided for validation at 1216. In this regard, a failed candidate is in substance a next iteration of the ‘selected implant model’ that was passed to the candidate implant specification module initially. For each iteration, the candidate implant model that did not pass is provided as the ‘selected implant model’ for a next iteration of the iterating, manipulations are optionally received, a next candidate is obtained, and it is provided to the validation module for validation. This iterating may be performed any number of times until a candidate is validated.

As an alternative to returning to 1214 after a candidate implant model has failed (1218, N), the process can instead return to 1220 to obtain a (next) selected implant model and work from that next model. This may particularly be the case in situations where the ML model identified more than one best-fit implant model. Additionally or alternatively, if the user has manipulated the patient anatomy as described above, it may be desired to reapply the ML model (returning to 1208) to see whether a different best-fit implant from the library is a better match.

By way of specific example in which a presented implant model is to be sent directly for validation (for instance when no manipulations are made and/or the selected implant model selected by the ML is provided directly to the validation as an initial task), the selected implant model is provided as a candidate implant model to the validation module for validation. In this case, the candidate implant model is an initial candidate implant model. Based on the validation determining that the physical implant specified by the initial candidate implant model does not pass (1218, N), the process returns to 1214. The process then receives at 1214 (i) manipulations to the initial candidate implant model, the manipulations changing the initial candidate implant model and producing a different candidate implant model for validation, where the different candidate implant model specifies a physical implant with a different one or more physical properties in comparison to the physical implant specified by the initial candidate implant model and/or (ii) manipulations to the properties of the at least one anatomical surface of the other anatomy. The process, as part of providing the candidate implant model 1216, determines a next candidate implant model to provide to the validation module for validation: based on having received manipulations to the initial candidate implant model, the next candidate implant model to provide is determined to be the different implant model produced from the manipulations to the initial candidate implant model. However, based on receiving manipulations to the properties of the at least one anatomical surface of the other anatomy and not receiving manipulations to the initial candidate implant model, the next candidate implant model is determined to be the initial candidate implant model. In this manner, the next candidate implant model may or may not be the initial candidate implant model that failed. As described, this next candidate implant model is provided (1216) to the validation module for validation to determine whether the physical implant specified by the next candidate implant model passes for surgical implantation within the patient and that validation is based at least in part on the received manipulations to the initial candidate implant model and/or the manipulations to the properties of the at least one anatomical surface.

If instead at 1218 it is determined that the candidate is validated (1218, Y), then the process provides (1220) the candidate implant model as a specification the physical implant to use. At that point, the physical implant can be obtained if accessible or fabricated if desired.

In situations where the candidate implant model is a manipulated version of selected implant model selected from the library and that manipulated version does not separately exist in the library, it can be added to the library as a legitimate implant model. Additionally or alternatively, that candidate implant model can be indicated in a training dataset as part of a training example that correlates the candidate implant model to the at least one anatomical surface of the other anatomy, in order to associate that candidate implant model as a model appropriate with that anatomy. Furthermore, even if the candidate implant model does already exist in the library but has not, in training examples, been associated what the particular patient anatomy for which it was validated (for instance the patient anatomy is not reflected in existing training examples and/or the user has manipulated actual patient anatomy to produce a hypothetical patient anatomy that is not yet reflected), then that candidate implant model can be indicated in the training dataset as part of a training example that correlates the candidate implant model to that patient anatomy to associate that candidate implant model as a model appropriate with that anatomy.

The terms “connect,” “connected,” “contact” “coupled” and/or the like are broadly defined herein to encompass a variety of divergent arrangements and assembly techniques. These arrangements and techniques include, but are not limited to (1) the direct joining of one component and another component with no intervening components therebetween (e.g., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (notwithstanding the presence of one or more additional components therebetween). It is to be understood that some components that are in direct physical contact with one another may or may not be in electrical contact and/or fluid contact with one another. Moreover, two components that are electrically connected, electrically coupled, optically connected, optically coupled, fluidly connected or fluidly coupled may or may not be in direct physical contact, and one or more other components may be positioned therebetween.

The terms “including” and “comprising”, as used herein, mean the same thing.

The terms “substantially”, “approximately”, “about”, “relatively,” or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, they can refer to less than or equal to +10%, such as less than or equal to +5%, such as less than or equal to +2%, such as less than or equal to +1%, such as less than or equal to +0.5%, such as less than or equal to +0.2%, such as less than or equal to +0.1%, such as less than or equal to +0.05%. If used herein, the terms “substantially”, “approximately”, “about”, “relatively,” or other such similar terms may also refer to no fluctuations.

As used herein, “electrically coupled” refers to a transfer of electrical energy between any combination of a power source, an electrode, a conductive surface, a droplet, a conductive trace, wire, waveguide, nanostructures, other circuit segment and the like. The terms electrically coupled may be utilized in connection with direct or indirect connections and may pass through various intermediaries, such as a fluid intermediary, an air gap and the like.

As used herein, “neural networks” refer to a biologically inspired programming paradigm which enable a computer to learn from observational data. This learning is referred to as deep learning, which is a set of techniques for learning in neural networks. Neural networks, including modular neural networks, are capable of pattern recognition with speed, accuracy, and efficiency, in situations where data sets are multiple and expansive, including across a distributed network of the technical environment. Modern neural networks are non-linear statistical data modeling tools. They are usually used to model complex relationships between inputs and outputs or to identify patterns in data (i.e., neural networks are non-linear statistical data modeling or decision-making tools). In general, program code utilizing neural networks can model complex relationships between inputs and outputs and identify patterns in data. Because of the speed and efficiency of neural networks, especially when parsing multiple complex data sets, neural networks and deep learning provide solutions to many problems in image recognition, speech recognition, and natural language processing. Neural networks can model complex relationships between inputs and outputs to identify patterns in data, including in images, for classification. Because of the speed and efficiency of neural networks, especially when parsing multiple complex data sets, neural networks and deep learning provide solutions to many problems in image recognition, which are not otherwise possible outside of this technology. As described below, the neural networks in some embodiments of the present invention are utilized to learn various features of various features of talus implants designs, including but not limited to, focusing on articular surfaces.

As used herein, a “convolutional neural network” (CNN) is a class of neural network. CNNs utilizes feed-forward artificial neural networks and are most commonly applied to analyzing visual imagery. CNNs are so named because they utilize convolutional layers that apply a convolution operation (a mathematical operation on two functions to produce a third function that expresses how the shape of one is modified by the other) to the input, passing the result to the next layer. The convolution emulates the response of an individual neuron to visual stimuli. Each convolutional neuron processes data only for its receptive field. It is not practical to utilize general (i.e., fully connected feedforward) neural networks to process images, as very high number of neurons would be necessary, due to the very large input sizes associated with images. Utilizing a CNN addresses this issue as it reduces the number of free parameters, allowing the network to be deeper with fewer parameters, as regardless of image size, the CNN can utilize a consistent number of learnable parameters because CNNs fine-tune large amounts of parameters and massive pre-labeled datasets to support a learning process. CNNs resolve the vanishing or exploding gradients problem in training traditional multi-layer neural networks, with many layers, by using backpropagation. Thus, CNNs can be utilized in large-scale (image) recognition systems, giving state-of-the-art results in segmentation, object detection and object retrieval. CNNs can be of any number of dimensions, but most existing CNNs are two-dimensional and process single images. These images contain pixels in a two-dimensional (2D) space (length, width) that are processed through a set of two-dimensional filters to understand what set of pixels best correspond to the final output classification. A three-dimensional CNN (3D-CNN) is an extension of the more traditional two-dimensional CNN and a 3D-CNN is typically used in problems related to video classification. 3D-CNNs accept multiple images, often sequential image frames of a video, and use 3D filters to understand the 3D set of pixels that are presented to it. In the present context, as discussed herein, images provided to a CNN include anatomical images of a patient.

As used herein, a “classifier” is comprised of various cognitive algorithms, artificial intelligence (AI) instruction sets, and/or machine learning algorithms. Classifiers can include, but are not limited to, deep learning models (e.g., neural networks having many layers) and random forests models. Classifiers classify items (data, metadata, objects, etc.) into groups, based on relationships between data elements in the metadata from the records. In some embodiments of the present invention, the program code can utilize the frequency of occurrences of features in mutual information to identify and filter out false positives. In general, program code utilizes a classifier to create a boundary between data of a first quality data of a second quality. As a classifier is continuously utilized, its accuracy can increase as testing the classifier tunes its accuracy. When training a classifier, in some examples, program code feeds a pre-existing feature set describing features of metadata and/or data into the one or more cognitive analysis algorithms that are being trained. The program code trains the classifier to classify records based on the presence or absence of a given condition, which is known before the tuning. The presence or absence of the condition is not noted explicitly in the records of the data set. When classifying a source as providing data of a given condition (based on the metadata), utilizing the classifier, the program code can indicate a probability of a given condition with a rating on a scale, for example, between 0 and 1, where 1 would indicate a definitive presence. The classifications need not be binary and can also be values in an established scale. As disclosed herein, a classifier is utilized, in some examples, to select an optimal talus from a database, based on program code cognitively analyzing patient anatomy.

As used herein, the term “deep learning model” refers to a type of classifier. A deep learning model can be implemented in various forms such as by a neural network (e.g., a convolutional neural network). In some examples, a deep learning model includes multiple layers, each layer comprising multiple processing nodes. In some examples, the layers process in sequence, with nodes of layers closer to the model input layer processing before nodes of layers closer to the model output. Thus, layers feeds to the next. Interior nodes are often “hidden” in the sense that their input and output values are not visible outside the model.

As used herein, the term “conditional generative adversarial network,” (“cGAN”), is a generative adversarial network (GAN), which is a machine learning framework, that is used to train generative models. Specifically, cGANs are utilized to conditionally generate images. GANs rely on a generator that learns to generate new images, and a discriminator that learns to distinguish synthetic images from real images. In cGANs, a conditional setting is applied, meaning that both the generator and discriminator are conditioned on auxiliary information from other modalities. Thus, a cGAN can learn multi-modal mapping from inputs to outputs by being fed with different contextual information. In the examples herein, contextual information can include both anatomical data from patients and a library of existing talus implants.

As used herein, the term “processor” refers to a hardware and/or software device that can execute computer instructions, including, but not limited to, one or more software processors, hardware processors, field programmable gate arrays (FPGAs), application-specific integrated circuits (ASICs), and/or programmable logic devices (PLDs).

Although various examples are provided, variations are possible without departing from a spirit of the claimed aspects.

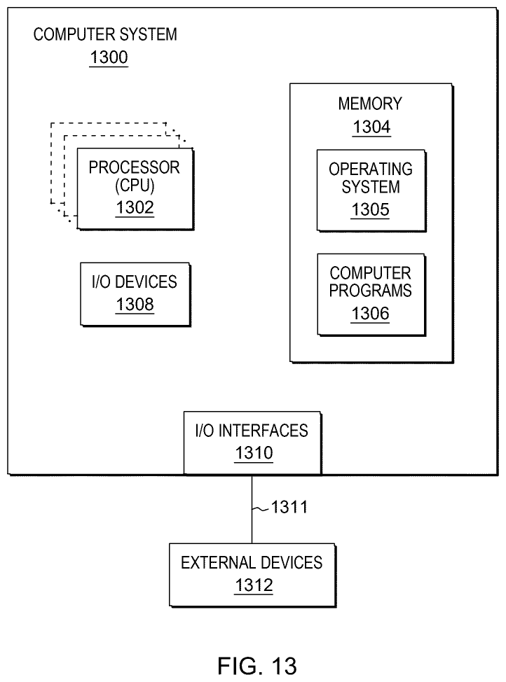

Processes described herein may be performed singly or collectively by one or more computer systems. FIG. 13 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 13 shows a computer system 1300 in communication with external device(s) 1312. Computer system 1300 includes one or more processor(s) 1302, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1302 can also include register(s) to be used by one or more of the functional components. Computer system 1300 also includes memory 1304, input/output (I/O) devices 1308, and I/O interfaces 1310, which may be coupled to processor(s) 1302 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1304 can be or include main or system memory (e.g. Random Access Memory) used in the execution of program instructions, storage device(s) such as hard drive(s), flash media, or optical media as examples, and/or cache memory, as examples. Memory 1304 can include, for instance, a cache, such as a shared cache, which may be coupled to local caches (examples include L1 cache, L2 cache, etc.) of processor(s) 1302. Additionally, memory 1304 may be or include at least one computer program product having a set (e.g., at least one) of program modules, instructions, code or the like that is/are configured to carry out functions of embodiments described herein when executed by one or more processors.

Memory 1304 can store an operating system 1305 and other computer programs 1306, such as one or more computer programs/applications that execute to perform aspects described herein. Specifically, programs/applications can include computer readable program instructions that may be configured to carry out functions of embodiments of aspects described herein.

Examples of I/O devices 1308 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (1312) coupled to the computer system through one or more I/O interfaces 1310.

Computer system 1300 may communicate with one or more external devices 1312 via one or more I/O interfaces 1310. Example external devices include a keyboard, a pointing device, a display, and/or any other devices that enable a user to interact with computer system 1300. Other example external devices include any device that enables computer system 1300 to communicate with one or more other computing systems or peripheral devices such as a printer. A network interface/adapter is an example I/O interface that enables computer system 1300 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1310 and external devices 1312 can occur across wired and/or wireless communications link(s) 1311, such as Ethernet-based wired or wireless connections. Example wireless connections include cellular, Wi-Fi, Bluetooth®, proximity-based, near-field, or other types of wireless connections. More generally, communications link(s) 1311 may be any appropriate wireless and/or wired communication link(s) for communicating data.

Particular external device(s) 1312 may include one or more data storage devices, which may store one or more programs, one or more computer readable program instructions, and/or data, etc. Computer system 1300 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1300 may be operational with numerous other general purpose or special purpose computing system environments or configurations. Computer system 1300 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
obtaining a machine learning model trained to select, based on properties of anatomical surfaces of anatomy that is adjacent to subject anatomy to be partially or totally replaced, anatomy implant models of physical implants for partial or total replacement of the subject anatomy;
obtaining imaging data of an anatomical region of a patient, the anatomical region comprising a subject anatomy of the patient and other anatomy of the patient, the other anatomy being adjacent to the subject anatomy of the patient;
determining, from the imaging data, properties of at least one anatomical surface of the other anatomy, the at least one anatomical surface being at a respective at least one interface between the other anatomy and the subject anatomy of the patient; and
applying the machine learning model, using the determined properties of the at least one anatomical surface, and obtaining, based on the applying, a selected implant model, the selected implant model being selected by the machine learning model as a specification of a physical implant for potential surgical implantation at least partially within the patient as a partial or total replacement of the subject anatomy of the patient.

2. The method of claim 1, further comprising providing the selected implant model to a candidate model specification module for specification of a candidate model for validation.

3. The method of claim 2, further comprising:
presenting the selected implant model to a user on a graphical user interface;
receiving manipulations to the selected implant model, the manipulations changing the selected implant model and producing the candidate implant model for validation, wherein the candidate implant model specifies a physical implant with a different one or more physical properties in comparison to the physical implant specified by the selected implant model; and
providing the candidate implant model to a validation module for validation, the validation determining whether the physical implant specified by the candidate implant model passes for surgical implantation within the patient.

4. The method of claim 3, wherein the manipulations to the selected implant model comprise at least one selected from the group consisting of:
at least one manipulation specified by the user; and
at least one manipulation determined automatically by artificial intelligence.

5. The method of claim 3, wherein based on the validation determining that the physical implant specified by the candidate implant model does not pass, the method further comprises iterating, one or more times, (i) the receiving manipulations and (ii) the providing the candidate implant model to the validation module for validation, wherein at each iteration of the iterating, the candidate implant model that did not pass is provided as the selected implant model for a next iteration of the iterating.

6. The method of claim 3, further comprising receiving manipulations to the properties of the at least one anatomical surface of the other anatomy, wherein the validation to determine whether the physical implant specified by the candidate implant model passes for surgical implantation within the patient is based at least in part on the manipulated properties of the at least one anatomical surface.

7. The method of claim 6, wherein the manipulations to the properties of the at least one anatomical surface comprise at least one selected from the group consisting of:
at least one manipulation specified by the user; and
at least one manipulation determined automatically by artificial intelligence.

8. The method of claim 2, further comprising:
receiving manipulations to the properties of the at least one anatomical surface of the other anatomy; and
providing the selected implant model, as the candidate implant model for validation, to a validation module for validation, the validation determining whether the physical implant specified by the candidate implant model passes for surgical implantation within the patient, wherein the validation is based at least in part on the manipulated properties of the at least one anatomical surface.

9. The method of claim 2, wherein based on determining the candidate implant model after receiving at least one selected from the group consisting of (i) manipulations to the selected implant model, the manipulations changing the selected implant model and producing the candidate implant model for validation, wherein the different candidate implant model specifies a physical implant with a different one or more physical properties in comparison to the physical implant specified by the selected implant model, and (ii) manipulations to the properties of the at least one anatomical surface of the other anatomy, the method further comprises:
providing the candidate implant model to a validation module for validation, the validation determining whether the physical implant specified by the candidate implant model passes for surgical implantation within the patient; and
based on the validation determining that the physical implant specified by the candidate implant model passes for surgical implantation within the patient, indicating the candidate implant model in a training dataset as part of a training example that correlates the candidate implant model to the at least one anatomical surface of the other anatomy.

10. The method of claim 1, further comprising providing the selected implant model, as a candidate implant model, to a validation module for validation, the validation determining whether the physical implant specified by the candidate implant model passes for surgical implantation within the patient.

11. The method of claim 10, wherein the candidate implant model is an initial candidate implant model, wherein based on the validation determining that the physical implant specified by the initial candidate implant model does not pass, the method further comprises:
receiving at least one selected from the group consisting of:
manipulations to the initial candidate implant model, the manipulations changing the initial candidate implant model and producing a different candidate implant model for validation, wherein the different candidate implant model specifies a physical implant with a different one or more physical properties in comparison to the physical implant specified by the initial candidate implant model; and
manipulations to the properties of the at least one anatomical surface of the other anatomy;
determining a next candidate implant model to provide to the validation module for validation, wherein:
based on receiving manipulations to the initial candidate implant model, the next candidate implant model is determined to be the different implant model produced from the manipulations to the initial candidate implant model; or
based on receiving manipulations to the properties of the at least one anatomical surface of the other anatomy and not receiving manipulations to the initial candidate implant model, the next candidate implant model is determined to be the initial candidate implant model; and
providing the next candidate implant model to the validation module for validation to determine whether the physical implant specified by the next candidate implant model passes for surgical implantation within the patient, wherein the validation to determine whether the physical implant specified by the next candidate implant model passes for surgical implantation within the patient is based at least in part on the received at least one selected from the group consisting of the manipulations to the initial candidate implant model and the manipulations to the properties of the at least one anatomical surface.

12. The method of claim 1, further comprising training the machine learning model to select the anatomy implant models, wherein the training uses samples from a library of implant models and trains the machine learning model to select the anatomy implant models from the library of implant models, and wherein the selected implant model is selected by the machine learning model from the library of implant models.

13. The method of claim 1, wherein the machine learning model comprises a trained generator of a generative adversarial network (GAN), wherein the generator is trained using samples from a library of implant models, and is trained to generate implant models, wherein the selected implant model comprises an implant model generated by the generator and selected by the generator for output as the selected implant model.

14. The method of claim 1, wherein the obtained imaging data comprises three-dimensional digital model data representing the anatomical region of the patient, and wherein the determining the properties of at least one anatomical surface of the other anatomy comprises:
processing the imaging data to present the at least one anatomical surface as at least one digital three-dimensional surface; and
converting the at least one digital three-dimensional surface to at least one two-dimensional projection, wherein the determined properties of the at least one anatomical surface are determined from the at least one two-dimensional projection.

15. The method of claim 14, further comprising preprocessing the imaging data to produce a three-dimensional digital model of the anatomical region of the patient with the subject anatomy omitted therefrom.

16. The method of claim 1, wherein the one or more anatomical surfaces comprise one or more articular surfaces with which the physical implant is to engage based on being surgically implanted at least partially within the patient.

17. The method of claim 1, wherein the anatomical region comprises a patient ankle, wherein the subject anatomy comprises a talus, and wherein the at least one anatomical surface comprises at least one articular surface of at least one bone adjacent to the talus.

18. The method of claim 1, wherein the determining the properties of the at least one anatomical surface is based on at least one selected from the group consisting of:
manual indication, by a user, of the at least one anatomical surface provided based on user input to a graphical user interface displaying a model comprising at least the other anatomy of the patient; and
automated analysis of the imaging data to ascertain the at least one anatomical surface.

19. A computer system comprising:
a memory; and
a processor in communication with the memory, wherein the computer system is configured to perform a method comprising:
obtaining a machine learning model trained to select, based on properties of anatomical surfaces of anatomy that is adjacent to subject anatomy to be partially or totally replaced, anatomy implant models of physical implants for partial or total replacement of the subject anatomy;
obtaining imaging data of an anatomical region of a patient, the anatomical region comprising a subject anatomy of the patient and other anatomy of the patient, the other anatomy being adjacent to the subject anatomy of the patient;
determining, from the imaging data, properties of at least one anatomical surface of the other anatomy, the at least one anatomical surface being at a respective at least one interface between the other anatomy and the subject anatomy of the patient; and
applying the machine learning model, using the determined properties of the at least one anatomical surface, and obtaining, based on the applying, a selected implant model, the selected implant model being selected by the machine learning model as a specification of a physical implant for potential surgical implantation at least partially within the patient as a partial or total replacement of the subject anatomy of the patient.

20. The computer system of claim 19, wherein the method further comprises providing the selected implant model to a candidate model specification module for specification of a candidate model for validation.

21. The computer system of claim 20, wherein the method further comprises:
presenting the selected implant model to a user on a graphical user interface;
receiving manipulations to the selected implant model, the manipulations changing the selected implant model and producing the candidate implant model for validation, wherein the candidate implant model specifies a physical implant with a different one or more physical properties in comparison to the physical implant specified by the selected implant model; and
providing the candidate implant model to a validation module for validation, the validation determining whether the physical implant specified by the candidate implant model passes for surgical implantation within the patient.

22. The computer system of claim 21, wherein the method further comprises receiving manipulations to the properties of the at least one anatomical surface of the other anatomy, wherein the validation to determine whether the physical implant specified by the candidate implant model passes for surgical implantation within the patient is based at least in part on the manipulated properties of the at least one anatomical surface.

23. The computer system of claim 20, wherein the method further comprises:
receiving manipulations to the properties of the at least one anatomical surface of the other anatomy; and
providing the selected implant model, as the candidate implant model for validation, to a validation module for validation, the validation determining whether the physical implant specified by the candidate implant model passes for surgical implantation within the patient, wherein the validation is based at least in part on the manipulated properties of the at least one anatomical surface.

24. The computer system of claim 19, wherein the method further comprises providing the selected implant model, as a candidate implant model, to a validation module for validation, the validation determining whether the physical implant specified by the candidate implant model passes for surgical implantation within the patient, wherein the candidate implant model is an initial candidate implant model, wherein based on the validation determining that the physical implant specified by the initial candidate implant model does not pass, the method further comprises:
receiving at least one selected from the group consisting of:
manipulations to the initial candidate implant model, the manipulations changing the initial candidate implant model and producing a different candidate implant model for validation, wherein the different candidate implant model specifies a physical implant with a different one or more physical properties in comparison to the physical implant specified by the initial candidate implant model; and
manipulations to the properties of the at least one anatomical surface of the other anatomy;
determining a next candidate implant model to provide to the validation module for validation, wherein:
based on receiving manipulations to the initial candidate implant model, the next candidate implant model is determined to be the different implant model produced from the manipulations to the initial candidate implant model; or
based on receiving manipulations to the properties of the at least one anatomical surface of the other anatomy and not receiving manipulations to the initial candidate implant model, the next candidate implant model is determined to be the initial candidate implant model; and
providing the next candidate implant model to the validation module for validation to determine whether the physical implant specified by the next candidate implant model passes for surgical implantation within the patient, wherein the validation to determine whether the physical implant specified by the next candidate implant model passes for surgical implantation within the patient is based at least in part on the received at least one selected from the group consisting of the manipulations to the initial candidate implant model and the manipulations to the properties of the at least one anatomical surface.

25. The computer system of claim 19, wherein the method further comprises preprocessing the imaging data to produce a three-dimensional digital model of the anatomical region of the patient with the subject anatomy omitted therefrom, wherein the obtained imaging data comprises three-dimensional digital model data representing the anatomical region of the patient, and wherein the determining the properties of at least one anatomical surface of the other anatomy comprises:
processing the imaging data to present the at least one anatomical surface as at least one digital three-dimensional surface; and
converting the at least one digital three-dimensional surface to at least one two-dimensional projection, wherein the determined properties of the at least one anatomical surface are determined from the at least one two-dimensional projection.

26. The computer system of claim 19, wherein the anatomical region comprises a patient ankle, wherein the subject anatomy comprises a talus, and wherein the at least one anatomical surface comprises at least one articular surface of at least one bone adjacent to the talus.

27. A computer program product comprising:
a computer readable storage medium readable by a processing circuit and storing instructions for execution by the processing circuit for performing a method comprising:
obtaining a machine learning model trained to select, based on properties of anatomical surfaces of anatomy that is adjacent to subject anatomy to be partially or totally replaced, anatomy implant models of physical implants for partial or total replacement of the subject anatomy;
obtaining imaging data of an anatomical region of a patient, the anatomical region comprising a subject anatomy of the patient and other anatomy of the patient, the other anatomy being adjacent to the subject anatomy of the patient;
determining, from the imaging data, properties of at least one anatomical surface of the other anatomy, the at least one anatomical surface being at a respective at least one interface between the other anatomy and the subject anatomy of the patient; and
applying the machine learning model, using the determined properties of the at least one anatomical surface, and obtaining, based on the applying, a selected implant model, the selected implant model being selected by the machine learning model as a specification of a physical implant for potential surgical implantation at least partially within the patient as a partial or total replacement of the subject anatomy of the patient.

28. The computer program product of claim 27, wherein the method further comprises providing the selected implant model to a candidate model specification module for specification of a candidate model for validation.

29. The computer program product of claim 28, wherein the method further comprises:
presenting the selected implant model to a user on a graphical user interface;
receiving manipulations to the selected implant model, the manipulations changing the selected implant model and producing the candidate implant model for validation, wherein the candidate implant model specifies a physical implant with a different one or more physical properties in comparison to the physical implant specified by the selected implant model; and
providing the candidate implant model to a validation module for validation, the validation determining whether the physical implant specified by the candidate implant model passes for surgical implantation within the patient.

30. The computer program product of claim 29, wherein the method further comprises receiving manipulations to the properties of the at least one anatomical surface of the other anatomy, wherein the validation to determine whether the physical implant specified by the candidate implant model passes for surgical implantation within the patient is based at least in part on the manipulated properties of the at least one anatomical surface.

31. The computer program product of claim 28, wherein the method further comprises:
receiving manipulations to the properties of the at least one anatomical surface of the other anatomy; and
providing the selected implant model, as the candidate implant model for validation, to a validation module for validation, the validation determining whether the physical implant specified by the candidate implant model passes for surgical implantation within the patient, wherein the validation is based at least in part on the manipulated properties of the at least one anatomical surface.

32. The computer program product of claim 27, wherein the method further comprises providing the selected implant model, as a candidate implant model, to a validation module for validation, the validation determining whether the physical implant specified by the candidate implant model passes for surgical implantation within the patient, wherein the candidate implant model is an initial candidate implant model, wherein based on the validation determining that the physical implant specified by the initial candidate implant model does not pass, the method further comprises:
receiving at least one selected from the group consisting of:
manipulations to the initial candidate implant model, the manipulations changing the initial candidate implant model and producing a different candidate implant model for validation, wherein the different candidate implant model specifies a physical implant with a different one or more physical properties in comparison to the physical implant specified by the initial candidate implant model; and
manipulations to the properties of the at least one anatomical surface of the other anatomy;
determining a next candidate implant model to provide to the validation module for validation, wherein:
based on receiving manipulations to the initial candidate implant model, the next candidate implant model is determined to be the different implant model produced from the manipulations to the initial candidate implant model; or
based on receiving manipulations to the properties of the at least one anatomical surface of the other anatomy and not receiving manipulations to the initial candidate implant model, the next candidate implant model is determined to be the initial candidate implant model; and
providing the next candidate implant model to the validation module for validation to determine whether the physical implant specified by the next candidate implant model passes for surgical implantation within the patient, wherein the validation to determine whether the physical implant specified by the next candidate implant model passes for surgical implantation within the patient is based at least in part on the received at least one selected from the group consisting of the manipulations to the initial candidate implant model and the manipulations to the properties of the at least one anatomical surface.

33. The computer program product of claim 27, wherein the obtained imaging data comprises three-dimensional digital model data representing the anatomical region of the patient, wherein the method further comprises preprocessing the imaging data to produce a three-dimensional digital model of the anatomical region of the patient with the subject anatomy omitted therefrom, and wherein the determining the properties of at least one anatomical surface of the other anatomy comprises:
processing the imaging data to present the at least one anatomical surface as at least one digital three-dimensional surface; and
converting the at least one digital three-dimensional surface to at least one two-dimensional projection, wherein the determined properties of the at least one anatomical surface are determined from the at least one two-dimensional projection.

34. The computer program product of claim 27, wherein the anatomical region comprises a patient ankle, wherein the subject anatomy comprises a talus, and wherein the at least one anatomical surface comprises at least one articular surface of at least one bone adjacent to the talus.
